(19) United States
(12) Patent Application Publication
(10) Pub. No.: US 2024/0157068 A1
(43) Pub. Date: May 16, 2024

(54) INHALATION ACTUATOR, INSERT AND INHALATION DEVICE

(71) Applicant: WERRTA GmbH Düsen- und Zerstäubungstechnik, Berlin (DE)

(72) Inventors: Rüdiger RENTSCH, Berlin (DE); Mathias ETZOLD, Berlin (DE)

(21) Appl. No.: 18/552,617

(22) PCT Filed: Mar. 29, 2022
(86) PCT No.: PCT/EP2022/058292

§ 371 Date: Sep. 26, 2023

(30) Foreign Application Priority Data
Apr. 15, 2021 (DE) 10 2021 203 756.9

(51) Int. Cl. A61M 15/00; B05B 1/26; B05B 1/28; B65D 83/30

(52) U.S. Cl. CPC A61M 15/009; A61M 15/0013; A61M 15/0021; B05B 1/262; B05B 1/28; B65D 83/30; A61M 2206/14

(57) ABSTRACT

Inhalation actuator (6), having a receptacle (5) for receiving a discharge pipe (4) of an inhalant reservoir (1), an inhalant nozzle (9) for spraying inhalant from at least one nozzle outlet opening, an inhalant delivery system (8) for delivering inhalant from the discharge pipe to the inhalant nozzle, a baffle element (10) onto which inhalant can be sprayed in a straight line from the nozzle outlet opening, an aerosol chamber (12) with an internal volume to which inhalant sprayed from the inhalant nozzle can be delivered, wherein the aerosol chamber has an aerosol outlet (11), which has a local narrowing, for dispensing aerosol from the aerosol chamber, and an air delivery system (13) separate from the inhalant nozzle and from the aerosol outlet. Inhalation device having the inhalant reservoir with a pressurized container (2), and having the inhalation actuator, wherein the inhalation actuator does not need to have the local narrowing. Insert (21) for inserting into the mouthpiece (18) of an inhalation actuator, the insert having: centring means for centring the insert in the mouthpiece, a wall (25) which is arranged transversely with respect to the insertion direction and which is arranged upstream of the centring means in the insertion direction, an inhalant inlet opening (24) provided in the wall, a baffle element, which is arranged upstream of the inhalant inlet opening counter to the insertion direction, a surface which is arranged transversely with respect to the insertion direction, next to the baffle element, and though which aerosol can flow freely in the insertion direction, an air inlet (13) arranged upstream of the free-flow surface in the insertion direction, and an aerosol outlet which is arranged upstream of the free-flow surface counter to the insertion direction.

The present invention relates to an inhalation actuator and an inhalation device comprising the inhalation actuator.

The part of an inhalation device which is handled by the user of the inhalation device in order to obtain inhalate for inhalation from an inhalate reservoir is referred to as inhalation actuator. If the inhalation device is a pressurized gas inhaler, also commonly referred to as an inhalation spray, inhalation nebulizer or metered dose inhaler (due to a limited amount of inhalant dispensed per spray burst), the inhalation actuator, which is usually designed as a unit consisting of a mouthpiece and a spray can receptacle, interacts with the inhalation reservoir, which is designed as a spray can, by the user pressing the spray can and the inhalation actuator against each other, for example.

A disadvantage of conventional pressurized gas inhalers is the high discharge speed of the aerosol (spray) formed from the inhalate, which is usually delivered from an inhalate nozzle through the mouthpiece directly in the direction of the mouth. The high discharge speed of the spray (in the magnitude of 2 to 10 m/s) and at the same time short discharge time (in the magnitude of 0.2 s) require a very controlled behavior of the user. Therein, coordinating the manual operation of the inhalation actuator with the breath becomes a decisive factor for the efficacy of the drug contained in the inhalate, which can determine the success of the therapy. For only if the short burst of spray takes place during a breath and neither too early nor too late can a sufficient proportion of the discharged inhalate also reach the user's lower airways.

To alleviate these problems for the user, so-called spacers are used as inhalation aids. This relates to a chamber to be arranged between the inhalation actuator and the user, in which discharged aerosol can accumulate, which the user draws out of the chamber by breathing in. The effect of the chamber is based on providing a relatively large volume of air in the chamber, which, together with the diffuser effect resulting from jet expansion, ensures that the aerosol ejected with the spray burst is slowed down. Such spacers are often perceived as awkward due to their size and cumbersome to handle, because they must first be attached to the mouthpiece of the inhalation actuator.

According to the invention, these prior art problems can be solved by integrating an aerosol chamber into the inhalation actuator and reducing the average droplet velocity in the inhalate ejected from an inhalate nozzle by using an impingement element. Therein, utilizing the impingement element permits a correspondingly compact design of the aerosol chamber, since no large volume of air is required to decelerate an aerosol stream in such an arrangement. The user can inhale the atomized inhalate from the aerosol chamber. The smaller droplet size due to the impingement can provide for greater stability of the aerosol in the aerosol chamber, i.e., in particular, less droplet precipitation on the inner walls of the aerosol chamber and a relatively low tendency of the aerosol droplets to coalesce with each other to form larger droplets.

In general, according to one aspect, the present invention provides an inhalation actuator comprising a receptacle for receiving a discharge nipple of an inhalate delivery device, an inhalate nozzle for spraying inhalate from at least one nozzle discharge opening, an inhalate feed for supplying inhalate from the discharge nipple to the inhalate nozzle, an impingement element onto which inhalate can be discharged in a straight line from the nozzle discharge opening, an aerosol chamber having an internal volume to which inhalate discharged from the inhalate nozzle can be supplied, the aerosol chamber comprising an aerosol outlet, preferably having a local constriction, for the discharge of aerosol from the aerosol chamber, and an air supply separate from the inhalate nozzle and the aerosol outlet.

According to another aspect, the invention provides an inhalation device comprising an inhalation actuator as described above and an inhalate reservoir, preferably a pressurized container. However, the invention can advantageously be implemented not only if the inhalate is discharged by the gas pressure of a spray can, but also using a hand pump, pressure generation by spring force or electrical pressure generation.

The pressure container may advantageously be equipped with a dosing valve, as is known per se, for example, from conventional metered dose sprays.

Advantageously, the discharge nipple of the inhalate reservoir may be oriented in the direction of a longitudinal axis of the pressure container, and at least the majority of the internal volume of the aerosol chamber may be disposed within a projection of the pressure container in the direction of its longitudinal axis. If the internal volume of the aerosol chamber is variable, the criterion of this embodiment applies to the maximum extent of the aerosol chamber, preferably to the maximum and minimum extent of the aerosol chamber.

According to a particularly advantageous aspect of the invention, especially with respect to achievable compactness, there is provided in particular an inhalation device comprising an inhalate reservoir comprising a pressure container having a longitudinal axis and a discharge nipple oriented in the direction of the longitudinal axis, and an inhalation actuator. The inhalation actuator comprises a receptacle for receiving the discharge nipple, an inhalate nozzle for discharging inhalate from at least one nozzle discharge opening, an inhalate feed for supplying inhalate from the discharge nipple to the inhalate nozzle, an impingement element onto which inhalate can be discharged in a straight line from the nozzle discharge opening, and an aerosol chamber having an internal volume, to which inhalate discharged from the inhalate nozzle can be supplied, wherein the aerosol chamber comprises an aerosol outlet for outflow of aerosol from the aerosol chamber and an air supply separate from the inhalate nozzle and the aerosol outlet, and wherein at least the major portion of the interior volume of the aerosol chamber is disposed within a projection of the pressure container in the direction of its longitudinal axis. If the internal volume of the aerosol chamber is variable, this criterion applies to the maximum expansion of the aerosol chamber, preferably to the maximum and minimum expansion of the aerosol chamber.

According to an advantageous embodiment of the inhalation actuator or inhalation device according to the invention, the inhalation actuator comprises an insert which forms at least one wall of the aerosol chamber and holds the impingement element. In an advantageous further development of this embodiment, the insert can form the (entire) aerosol chamber. By means of an insert according to this embodiment or its further development, a conventional inhalation actuator can be converted in accordance with the invention.

According to an alternative advantageous embodiment of the inhalate actuator or inhalation device according to the invention, the inhalate feed and the aerosol chamber are formed integral with each other, which facilitates a compact design and safe handling.

Preferably, in an inhalation actuator according to the invention or in an inhalation device according to the invention, the at least one nozzle discharge opening and the aerosol outlet are arranged relative to each other in such a way that inhalate cannot flow in a straight line from the nozzle discharge opening to the aerosol outlet.

According to advantageous embodiments of the inhalation actuator according to the invention or of the inhalation device according to the invention, the main discharge direction of the at least one nozzle discharge opening and the main discharge direction of the aerosol outlet are arranged offset from one another, and/or the main discharge direction of the at least one nozzle discharge opening and the main discharge direction of the aerosol outlet are at an angle greater than zero, preferably greater than 29° and preferably less than 180°, particularly preferably less than 151° to one another. The main discharge direction is defined as a perpendicular to the center of area of the smallest flowable area of the nozzle discharge opening or the aerosol outlet, respectively.

Preferably, in an inhalation actuator according to the invention or in an inhalation device according to the invention, the air supply is provided with an air supply valve, for example a so-called flutter valve.

According to a further advantageous embodiment of the inhalation actuator according to the invention or of the inhalation device according to the invention, the aerosol outlet is equipped with an aerosol outlet valve.

According to a further advantageous embodiment of the inhalation actuator or inhalation device according to the invention, the inhalation actuator comprises a mouthpiece, wherein the aerosol outlet is preferably arranged between the aerosol chamber and the mouthpiece. A variant in which the inhalation actuator comprises an insert which forms at least one wall of the aerosol chamber or the entire aerosol chamber and holds the impingement element can advantageously be designed such that the insert can be inserted into the mouthpiece or through the mouthpiece.

Irrespective of the design of an aerosol chamber according to the invention with an aerosol outlet comprising a local constriction, an insert carrying an impingement element can advantageously be used to provide an inhalation actuator with an impingement element, in particular also with a configuration, according to which inhalate emerging from at least one nozzle discharge opening disintegrates into droplets before impinging on the impingement element (free jet disintegration into droplets, in particular also independently of additional gas flows), rather than a continuous inhalate jet being atomized not before reaching the impingement element, as described later herein.

According to another aspect of the invention, there is provided an insert for insertion into the mouthpiece of an inhalation actuator, the insert comprising: centering means for centering the insert in the mouthpiece, a wall disposed transverse to the direction of insertion and disposed in front of the centering means in the direction of insertion, an inhalant inlet opening provided in the wall, an impingement element which is arranged in front of the inhalant inlet opening in the direction opposite to the insertion direction, a through-flow area arranged next to the impingement element in the direction opposite to the insertion direction, an air inlet arranged in front of the through-flow area in the insertion direction, and an aerosol outlet arranged in front of the through-flow area in the direction opposite to the insertion direction. According to an advantageous embodiment of the insert, the centering means are configured integrally with a continuous or interrupted tube, wherein the longitudinal direction of the tube corresponds to the insertion direction. Opposite to the insertion direction, the wall and the tube thus form a pot-, chamber- or basket-like space in which the impingement element is located. Advantageously, the tube may comprise a rim narrowing the aerosol outlet on its side opposite the wall in the longitudinal direction. The air inlet may be at least partially formed as an interruption in the tube and/or at least partially formed in the wall. According to an advantageous embodiment of the insert, the cross-sectional area of the aerosol outlet that can be flowed through in the insertion direction (outlet area) is smaller than the through-flow area beside the impingement element. According to a further advantageous embodiment of the insert, it comprises an inhalant inlet neck upstream of the inhalant inlet opening in the direction of insertion.

The insert is preferably inserted into the mouthpiece of an inhalation actuator having a receptacle for receiving a discharge nipple of an inhalate delivery device, an inhalate nozzle for discharging inhalate from at least one nozzle discharge opening, an inhalate feed for supplying inhalate from the discharge nipple to the inhalate nozzle, and the mouthpiece arranged in front of the at least one nozzle discharge opening, such that a straight flow path exists from the at least one nozzle discharge opening through the inhalate inlet opening to the impingement element.

According to an advantageous further development of the inhalation actuator according to the invention or of the inhalation device according to the invention, the inhalation actuator comprises a collecting device for collecting inhalate that drips or flows off the impingement element and/or condenses in the aerosol chamber. A variant in which the inhalation actuator comprises an insert that forms at least one wall of the aerosol chamber or the entire aerosol chamber and supports the impingement element may advantageously be configured to also form the collecting device or a part of the collecting device.

Preferably, in an inhalation actuator according to the invention or in an inhalation device according to the invention, the collecting device comprises an absorbent piece of material, for example a fleece, sponge, zeolite or the like.

According to an advantageous embodiment of the inhalation actuator according to the invention or of the inhalation device according to the invention, the maximum extension of the inhalation actuator extends parallel to the discharge nipple inserted into the receptacle as intended.

According to a particularly advantageous embodiment of the inhalation actuator according to the invention or of the inhalation device according to the invention, in particular with regard to the compactness that can be achieved, at least 10 percent, preferably at least 25 percent, of the internal volume of the aerosol chamber is arranged behind the (or the respective) nozzle discharge opening with respect to a direction defined by a straight connecting line from the nozzle discharge opening (or at least one of the nozzle discharge openings) to the center of area of the discharge area of the aerosol outlet. If the internal volume of the aerosol chamber is variable, the criterion of this embodiment applies to the maximum expansion of the aerosol chamber, preferably to the maximum and the minimum expansion of the aerosol chamber.

Generally, in the context of the present invention, the location of the smallest area of the aerosol outlet through which the aerosol can flow is considered the outlet area. If this location is not clearly defined, for example, if the aerosol outlet is tubular or comprises no local constriction (for example, the entire aerosol chamber is designed as a tube open on one side), then among the smallest areas of the aerosol outlet through which the aerosol can flow, the one furthest from the user is the outlet area. The total volume through which the aerosol can flow between the nozzle discharge opening(s) and the outlet area of the aerosol outlet is included in the internal volume of the aerosol chamber.

Preferably, an inhalation device according to the invention is configured in such a way that inhalate emerging from the at least one nozzle discharge opening disintegrates into droplets before impinging on the impingement element (free jet disintegration into droplets, in particular also independently of additional gas flows), rather than a continuous inhalate jet first atomizing at the impingement element. Therein, the jet disintegration can be imagined in such a way that from a certain distance from the nozzle discharge opening, a straight-line chain of droplets is formed from a liquid jet emerging from the inhalate nozzle.

Such a design can be carried out empirically using simple design experiments. The skilled person can be guided by the following relationship

$$Z = D ln \frac{D}{2 C} \sqrt{We} [ 1 + 3 Oh ]$$

for jet break-up distance Z, wherein

$$We = \frac{ρ {U}^{2} D}{σ}$$

denotes the Weber number and

$$Oh = \frac{η}{\sqrt{D σ ρ}}$$

denotes the Ohnesorge number.
with
Z jet break-up distance in m
D narrowest nozzle diameter in m
C initial perturbation of the jet break-up in m
ρ density of the physiologically active liquid in kg/m³
σ surface tension of the physiologically active liquid in N/m
η viscosity of the physiologically active liquid in Pa s
U discharge velocity of the liquid jet from the nozzle

The initial perturbation of the jet break-up C is usually unknown, but it has turned out for the present invention that a value of the dimensionless factor

$$ln \frac{2 C}{D}$$

can usually be assumed to amount between 10 and 15, mostly from 12 to 13.

In the case of impact atomization of droplets formed by free jet break-up in a device according to the invention are, according to experimental values, for example

With a nozzle diameter of D=20 μm and pressures from 15 to 25 bar:
Dᵥ₉₀≈10 to 13 μm
Dᵥ₅₀≈6 to 8 μm
Dᵥ₁₀≈2.5 to 4 μm

With a nozzle diameter of D=15 μm and pressures from 15 to 25 bar:
Dᵥ₉₀≈8 to 10 μm
Dᵥ₅₀≈5 to 7 μm
Dᵥ₁₀≈2.5 to 4 μm

The diameter specifications in the above examples are to be understood as follows:
Dᵥ₁₀ 10% of the liquid volume of the aerosol consists of droplets smaller than Dᵥ₁₀
Dᵥ₅₀ 50% of the liquid volume of the aerosol consists of droplets smaller than Dᵥ₅₀
Dᵥ₉₀ 90% of the liquid volume of the aerosol consists of droplets smaller than Dᵥ₉₀

Physically, the process of an impact atomization of droplets created by free jet disintegration can be understood as follows: The atomization mechanism is less like the macroscopic process of a single large droplet hitting an obstacle, but is best described by considering two droplets hitting the same spot in succession. One impinging droplet forms a film on the impingement element, into which a subsequent droplet impacts, forming a “crown” from which smaller droplets then detach. Residual liquid remaining in the film can then in turn be impacted by a subsequent droplet resulting from free jet disintegration, forming a new crown from which smaller droplets again detach, and so on.

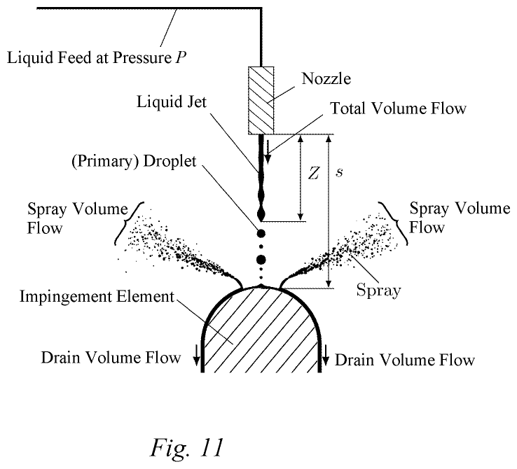

FIG. 11 further illustrates the process of impact atomization of droplets formed by free jet disintegration. A jet of liquid emerges from the nozzle and is supplied to the nozzle under pressure. After the jet breakup length Z, the liquid jet disintegrates into primary droplets, which successively impinge at (approximately) the same location on the impingement element, which is located at a distance s greater than the jet breakup length Z, opposite the nozzle opening. Following the process described above, each newly impinging primary droplet causes secondary droplets to detach from the liquid film on the impingement element, forming a spray volume flow. Part of the liquid runs off the impingement element. Efficient impact atomization is therefore not achieved by spraying a broad aerosol jet or spray cone onto a surface so that only randomly isolated droplets impinge on a spot on which a droplet has already impinged shortly before, but rather the concept of effective impact atomization is based on the fact that a stream of closely successive droplets, in particular those resulting from free jet disintegration, impinges on (approximately) the same spot.

In the context of the present invention, the inhalate is preferably provided as a liquid or suspension and is also supplied to the inhalate nozzle as a liquid or suspension, i.e., the inhalate nozzle is preferably not fed with an aerosol or a liquid or suspension with propellant gas dissolved therein respectively. When a suspension is supplied as an inhalate, such an embodiment ensures that static charging of the suspended particles is reduced or avoided. The suspended particles remain in the droplets of suspension liquid without being deposited to any great extent on the inner walls of the aerosol chamber.

The invention is explained in more detail below in an exemplary manner with reference to the attached schematic drawings. The drawings are not to scale; in particular, for reasons of clarity, the ratios of the individual dimensions to one another may not correspond in part to the dimensional ratios in actual technical implementations. Several preferred embodiments are described, but the invention is not limited thereto.

Generally, any variation of the invention described or suggested within the scope of the present application may be particularly advantageous, depending on the economic, technical and possibly medical conditions in the individual case. As far as nothing is stated to the contrary, or as far as generally technically feasible, individual features of the described embodiments can be interchanged or combined with each other as well as with features known per se from the prior art.

Therein

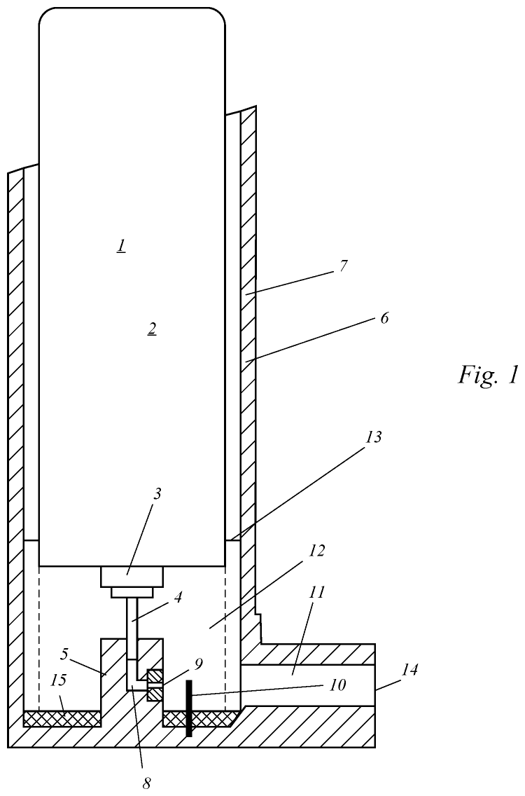

FIG. 1 shows in cross-section an inhalation device according to the invention with a pressure container as inhalate reservoir and an inhalation actuator according to the invention with an absorbent fleece for receiving inhalate dripping from the impingement element,

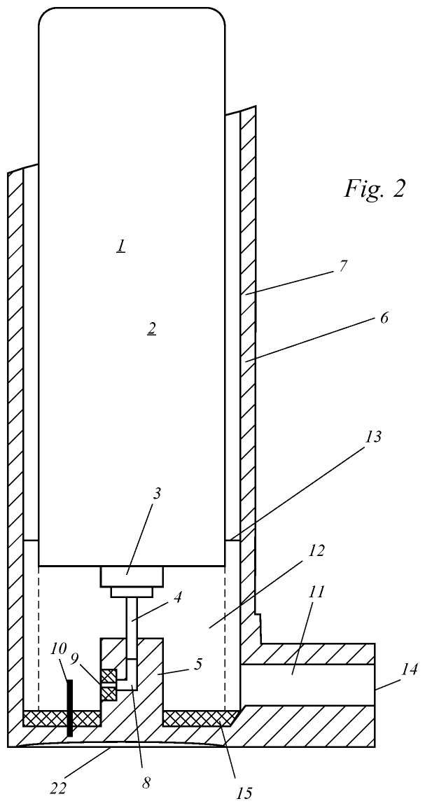

FIG. 2 shows in cross-section an inhalation device according to the invention like FIG. 1, in contrast to which, however, the nozzle discharge opening points in the opposite direction to the aerosol discharge,

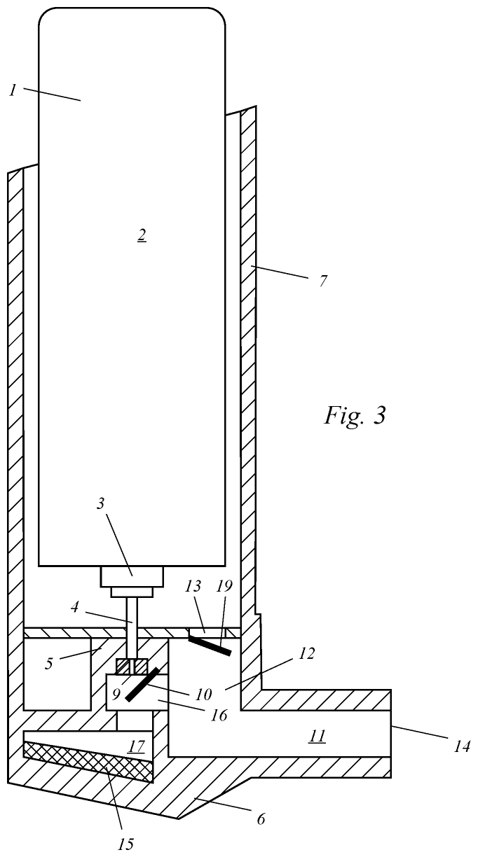

FIG. 3 shows in cross-section an inhalation device according to the invention having a pressure container as an inhalation reservoir and an inhalation actuator according to the invention, the aerosol chamber of which comprises an air supply with a flutter valve,

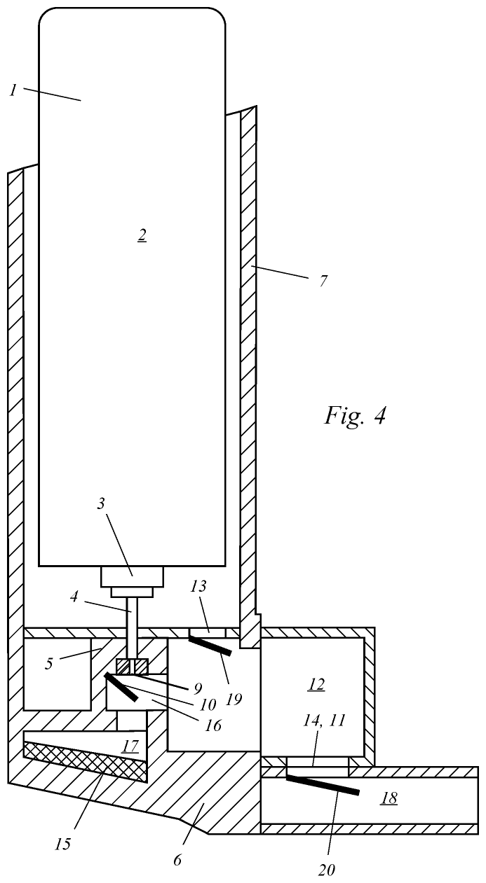

FIG. 4 shows a cross-section of an inhalation device according to the invention similar to FIG. 3 with a mouthpiece separate from the aerosol chamber and an arrangement of the impingement element that is different from FIG. 3,

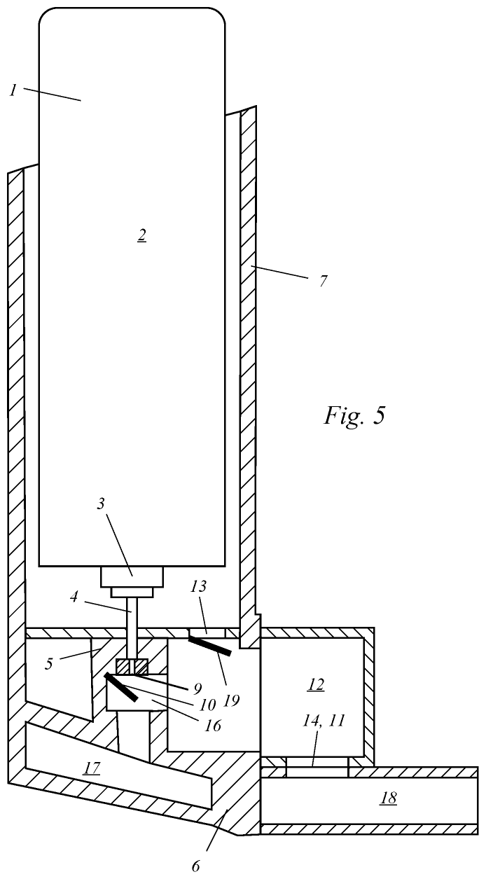

FIG. 5 shows in cross-section an inhalation device according to the invention like FIG. 4, wherein a collecting chamber for receiving inhalate dripping from the impingement element is provided instead of an absorbent fleece and a flutter valve at the aerosol outlet is omitted,

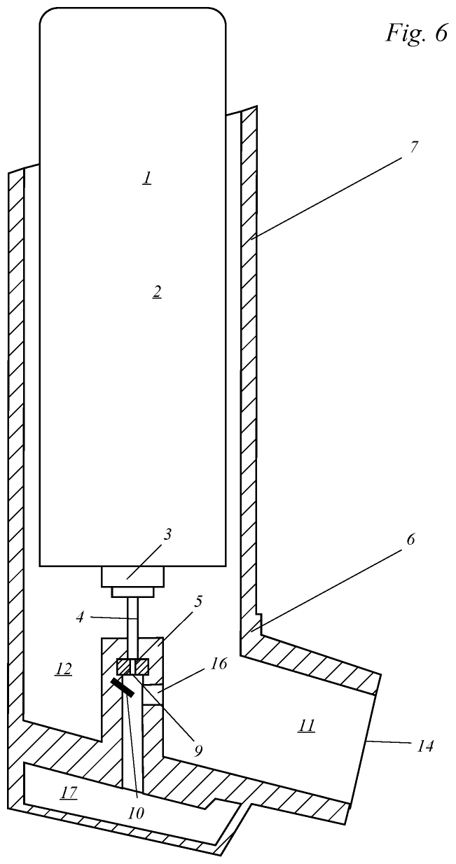

FIG. 6 shows in cross-section a simple design of an inhalation device according to the invention without a flutter valve and with a collection chamber for collecting inhalate dripping from the impingement element,

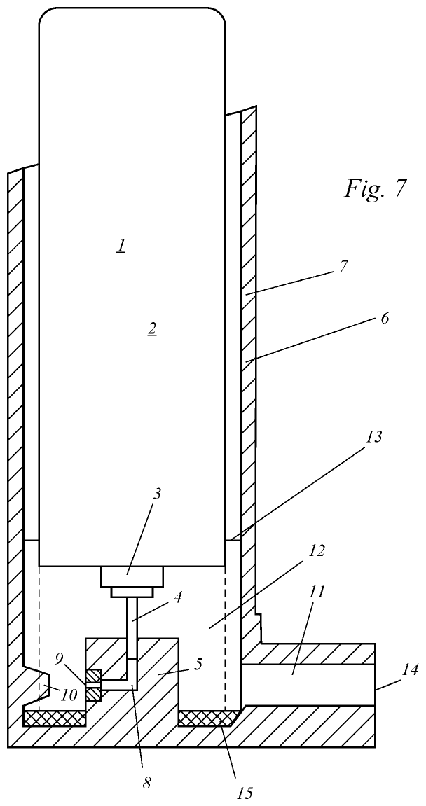

FIG. 7 shows in cross-section an inhalation device according to the invention like FIG. 2, in contrast to which the impingement element is formed as a projection in the wall of the aerosol chamber,

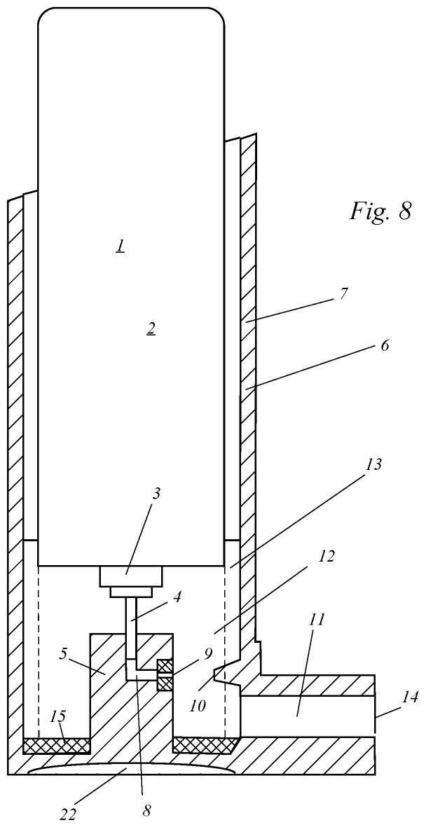

FIG. 8 shows in cross-section an inhalation device according to the invention like FIG. 1, in contrast to which the inhalate nozzle is slightly offset towards the pressure container and the impingement element is configured as a projection in the wall of the aerosol chamber,

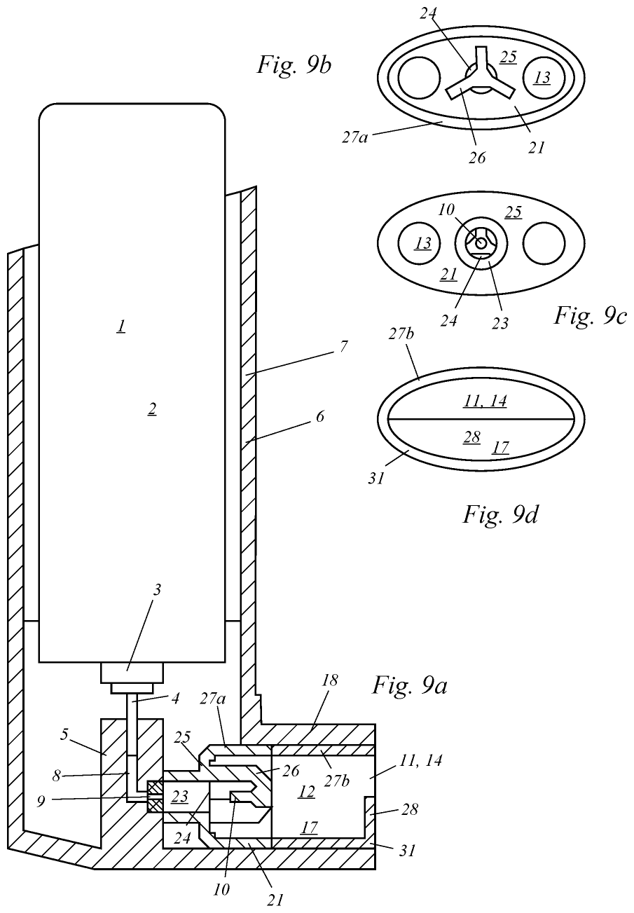

FIG. 9a shows in cross-section an inhalation device according to the invention, in which the impingement element is arranged on an insert, and the local constriction of the aerosol outlet is arranged on a further insert,

FIG. 9b shows a front view of the insert according to the invention of FIG. 9a, on which the impingement element is arranged (viewed from the right in FIG. 9a),

FIG. 9c shows the insert of FIG. 9b in rear view,

FIG. 9d shows the insert of FIG. 9a, on which the local constriction of the aerosol outlet is arranged, in a view from the left in FIG. 9a,

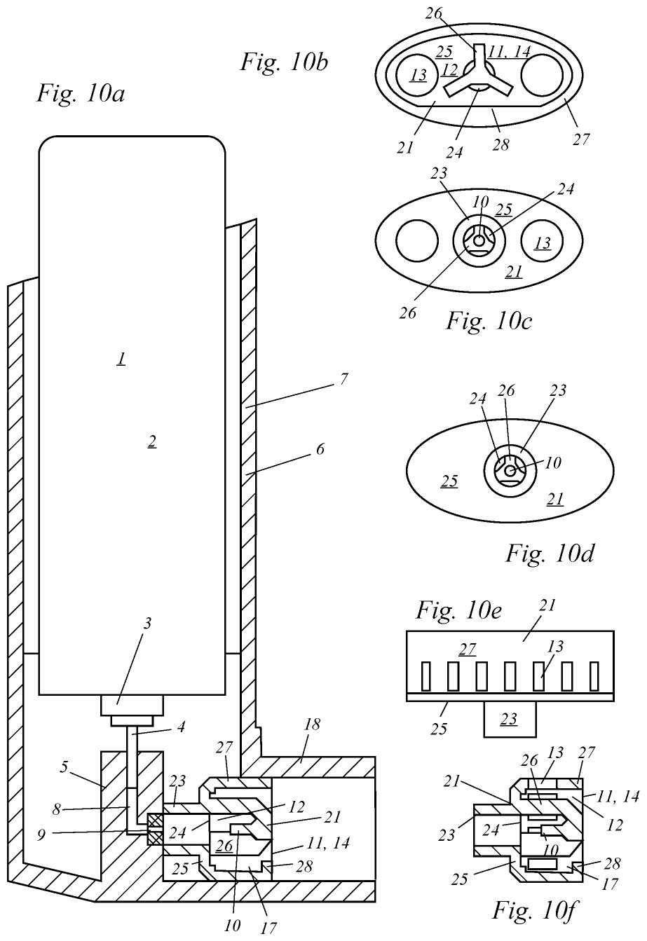

FIG. 10a shows in cross-section an inhalation device according to the invention similar to FIG. 9a, in which, however, the impingement element and the local constriction of the aerosol outlet are arranged in a joint insert,

FIG. 10b shows a front view of the insert according to the invention of FIG. 10a (viewed from the right in FIG. 10a),

FIG. 10c shows the insert of FIG. 10b in rear view,

FIG. 10d shows a rear view of an alternative insert according to the invention similar to FIG. 10c,

FIG. 10e shows a side view of the alternative insert of FIG. 10d, corresponding to a top or bottom view in FIG. 10f,

FIG. 10f shows the alternative insert of FIGS. 10d and 10d in a cross-sectional view corresponding to FIG. 10a, and

FIG. 11 shows an illustration of the impact atomization process.

In the individual figures, corresponding elements are marked with the respective same reference signs.

The inhalate reservoir 1 is designed like a conventional dosing spray from the medical field of application and comprises a pressure container 2, a metering valve unit 3 and a discharge nipple 4. Inhalate can emerge from the pressure container 2 through the discharge nipple 4 when the discharge nipple 4 is pressed axially into the metering valve unit 3. Pressing the discharge nipple 4, which is inserted into the receptacle 5 of the inhalation actuator 6, into the metering valve unit 3 is achieved by pressing the inhalation actuator 6 (hereinafter also referred to as actuator 6) and the pressure container 2 towards each other. For this purpose, the user can grip the sleeve 7 of the actuator 6 with the heel of the hand and the remaining four fingers of one hand, while pressing the pressure container 2 downward with the thumb against the spring force of a return spring (not shown) provided in the metering valve unit 3, thus triggering a spray burst. After the thumb pressure is released, the pressure container 2 is returned to its initial position by the return spring, and the device is ready for actuation for another spray burst. Preferably, however, the entire assembly is held between the thumb and forefinger and compressed, with the thumb at the bottom of the actuator 6 and the forefinger at the top of the bottom of the pressure container 2. For support, a thumb recess 22 can advantageously be provided herein in the head portion of the actuator 6.

Inhalate which is discharged during a spray burst passes through the inhalate feed 8 to the inhalate nozzle 9. The inhalate nozzle 9 is shown in the drawings as a nozzle body for example drilled by laser technology and inserted into the receptacle 5. However, the inhalate nozzle can also be formed integrally with the receptacle. The pressure gradient caused by the propellant pressure of the pressure container drives the liquid inhalate through the inhalate nozzle 9, from the nozzle discharge opening of which it is sprayed against the impingement element 10.

The propellant pressure in the pressure container 2, the diameter of the nozzle discharge opening and the distance between the nozzle discharge opening and the impingement element 10 are matched to each other and to the physical properties of the inhalate in such a way that, after leaving the nozzle discharge opening, the inhalate liquid breaks up into droplets by free jet disintegration, which then impinge on the impingement element 10. The impingement on the impingement element 10 further atomizes and decelerates the inhalate droplets. The droplet size distribution of the resulting aerosol is shifted towards smaller droplet diameters compared to the droplets created by free jet disintegration. This makes it possible to achieve good lung mobility for the inhalate. Excess inhalate dripping from the impingement element 10 is absorbed by the absorbent piece of material (fleece).

The fact that the impingement element 10 interrupts the direct connection between the nozzle discharge opening and the aerosol outlet 11, together with the deceleration of the inhalate droplets, ensures that inhalate is not already ejected from the aerosol outlet 11 at high velocity due to the pressure gradient at the inhalate nozzle. Instead, the aerosol produced during the spray burst can accumulate in the inner volume of the aerosol chamber 12, which is integrally designed with the receptacle.

The user can inhale the aerosol from the aerosol chamber 12 through the aerosol outlet 11, which is narrower than the aerosol chamber 12. For this purpose, the aerosol chamber 12 has an air supply 13 separate from the inhalate nozzle 9 and the aerosol outlet 11, which in the embodiment shown is formed by the seal arranged in the gap between the pressure container 2 and the envelope 7 of the actuator 6 being of porous structure.

The greater part, i.e. more than 50%, of the internal volume of the aerosol chamber 12 lies within the projection of the pressure container 2 in the direction of its longitudinal axis, as indicated by broken lines in FIG. 1, at least when the return spring in the metering valve unit 3 is released and the aerosol chamber 12 is at its maximum extent. The internal volume of the aerosol chamber includes the flowable volume in the aerosol outlet 11 as far as the outlet area 14. The outlet area 14 is the area furthest to the user with the smallest flowable area of the aerosol outlet 11. Since in the present embodiment the flowable area of the tubular aerosol outlet 11 is constant over a larger portion, the outlet area 14 is located on the far right in the figure.

More than 25 percent of the internal volume of the aerosol chamber 12 is located behind the nozzle discharge opening, with reference to the direction defined by a straight connecting line from the nozzle discharge opening of the inhalate nozzle 9 to the center of area of the discharge area 14 of the aerosol discharge 11, when the return spring in the metering valve unit 3 is released, but also still when the actuator 6 and pressure container 2 are maximally pressed towards each other.

Compared to conventional spacers, the aerosol chamber 12 is configured to be highly compact, so that it can be integrated into the actuator 6 and its maximum extension nevertheless extends parallel to the discharge nipple 4 inserted into the receptacle 5, i.e. the maximum extension of the inhalation device in the direction of the longitudinal axis of the pressure container 2.

The inhalation device in FIG. 2 is configured essentially the same as the inhalation device in FIG. 1. However, the main discharge direction of the nozzle discharge opening of the inhalate nozzle 9 is arranged at an angle 180° to the main discharge direction of the aerosol outlet 11.

The main discharge direction of the aerosol discharge 11 is orthogonal to the center of area of the discharge area 14, and the main discharge direction of the nozzle discharge opening is orthogonal to the center of area of the smallest flowable area of the nozzle discharge opening.

More than 10 percent of the internal volume of the aerosol chamber 12, with respect to the direction defined by a straight connecting line from the nozzle discharge opening of the inhalate nozzle 9 to the center of area of the discharge area 14 of the aerosol discharge opening 11, is located behind the nozzle discharge opening, namely when the return spring in the metering valve unit 3 is released, but also still when the actuator 6 and the pressure container 2 are maximally pressed towards each other.

The inhalation device in FIG. 3 also comprises an inhalate reservoir 1 having a pressure container 2, a metering valve unit 3 and a discharge nipple 4. The inhalate feed in the receptacle 5 of the actuator 6 is very short; the inhalate nozzle 9 is located practically directly at the end of the discharge nipple 4 opposite the metering valve unit 3, so that the nozzle discharge opening is arranged approximately coaxially to the discharge nipple 4. From the nozzle discharge opening, the inhalate passes into a small pre-chamber 16 in fluid communication with the aerosol chamber 12, in which the impingement element 10 is arranged. The surface of the impingement element 10 is at an angle of about 45° to the main discharge direction of the nozzle discharge opening between the nozzle discharge opening and the fluidic connection of the pre-chamber 16 to the aerosol chamber 12. This arrangement is used to further decelerate the inhalate.

Below the pre-chamber 16 and in fluidic communication herewith the inhalate collection chamber 17 is arranged, which contains absorbent material 15, for example made of fleece or silicate.

The aerosol outlet 11, through which atomized inhalate can be inhaled from the aerosol chamber 12, is in turn constricted relative to the aerosol chamber 12. The tubular aerosol outlet 11 can also serve as a mouthpiece that can be enclosed by the user's lips, or as an adapter for connecting a mask or the like.

The air supply 13, which is separate from the inhalation nozzle 9 and the aerosol outlet 11, is provided with a flap valve or flutter valve 19.

The greater part, i.e. more than 50%, of the internal volume of the aerosol chamber 12 is again within the projection of the pressure container 2 in the direction of its longitudinal axis. The internal volume of the aerosol chamber includes the flowable volume in the aerosol outlet 11 all the way to the outlet area 14.

Again, compared to conventional spacers, the aerosol chamber 12 is designed to be highly compact, so that it can be integrated into the actuator 6 and its maximum extension nevertheless extends parallel to the discharge nipple 4 inserted into the receptacle 5, i.e. the maximum extension of the inhalation device in the direction of the longitudinal axis of the pressure container 2.

The inhalation device in FIG. 4 is configured similarly to FIG. 3. It also comprises an inhalation device 1 with pressure container 2, metering valve unit 3 and discharge nipple 4. The metering valve unit 3 can again be operated by either gripping the sleeve 7 and pressing the pressure container 2 downward with the thumb, or preferably by holding the entire assembly between the thumb and index finger and pressing it together, the thumb being placed at the bottom of the actuator and the index finger at the top of the base of the pressure container 2. For support, a thumb recess (not shown) can advantageously be provided herein in the head portion of the actuator 6.

The inhalate feed 8 in the receptacle 5 of the actuator 6 coincides with the inlet of the inhalate nozzle 9, which is located directly at the end of the discharge nipple 4 opposite the metering valve unit 3, so that the nozzle discharge opening is arranged approximately coaxially with the discharge nipple 4. From the nozzle discharge opening, the inhalate passes into a small pre-chamber 16 in fluid communication with the aerosol chamber 12, in which the impingement element 10 is arranged. The surface of the impingement element 10 is at an angle of about 45° to the main discharge direction of the nozzle discharge opening as well as to the opening of the connection of the pre-chamber 16 into the aerosol chamber 12.

Below the pre-chamber 16 and in fluid communication therewith the inhalate collecting chamber 17 is arranged, which contains absorbent material 15, for example of fleece or silicate.

Constricted with respect to the aerosol chamber 12 is again the aerosol outlet 11, through which atomized inhalate can pass from the aerosol chamber 12 and be further inhaled through the mouthpiece 18. The main discharge direction of the aerosol outlet 11 is arranged approximately parallel to the main discharge direction of the nozzle discharge opening, but perpendicular to the main discharge opening of the connecting opening between the pre-chamber 16 and the aerosol chamber 12.

The air supply 13, which is separate from the inhalate nozzle 9 and the aerosol outlet 11, is provided with a flap valve or flutter valve 19.

The aerosol outlet 11 is also provided with a flutter valve 20.

The inhalation device in FIG. 5 is largely the same as in FIG. 4, but instead of the inhalate collection chamber in FIG. 4, which contains absorbent material, the inhalate collection chamber 17 is larger and designed without absorbent material.

The particularly simply designed inhalation device in FIG. 6 also has an inhalate collection chamber 17 without absorbent material.

The inhalate reservoir 1 comprises the pressure container 2, the dosing valve unit 3 and the discharge nipple 4. The metering valve unit 3 can in turn be operated by gripping the sleeve 7 and pressing the pressure container 2 downwards against the actuator 6 with the thumb, or preferably by holding the entire assembly between the thumb and index finger and pressing it together, the thumb being placed at the bottom of the actuator and the index finger at the top of the base of the pressure container 2. For support, a thumb recess can advantageously be provided herein in the head portion of the actuator 6.

The inhalate feed 8 in the receptacle 5 of the actuator 6 coincides with the inlet of the inhalate nozzle 9, which is located directly at the end of the discharge nipple 4 opposite the metering valve unit 3, so that the nozzle discharge opening is arranged approximately coaxially with the discharge nipple 4.

Like in FIG. 5, the inhalate passes from the nozzle discharge opening into a small pre-chamber 16 in fluid communication with the aerosol chamber 12, in which the impingement element 10 is arranged. The surface of the impingement element 10 is at an angle of about 45° to the main discharge direction of the nozzle discharge opening as well as to the opening of the connection of the pre-chamber 16 into the aerosol chamber 12.

The air inlet 13 into the aerosol chamber 12 is formed by the annular gap between actuator shell 7 and pressure container 2. The lower end of the pressure container 2 in this illustration also functions as the upper wall of the aerosol chamber 12 in this illustration.

Constricted with respect to the aerosol chamber 12 is again the aerosol outlet 11, through which atomized inhalate can be inhaled from the aerosol chamber 12. The aerosol outlet 11 in turn serves as a mouthpiece which the user can enclose with his lips.

While the impingement element 10 in FIGS. 1-6 is manufactured as a separate component, for example from a plastic, metallic or ceramic material, and is inserted into the actuator 6, the impingement element 10 may also be integrally formed. Accordingly, FIG. 7 shows an inhalation device corresponding to the greatest possible extent to FIG. 2, but in which the impingement element 10 is formed as a protrusion of the wall of the aerosol chamber 12. The inhalation device in FIG. 8 corresponds largely to the inhalation device in FIG. 1, but herein the impingement element 10 is also formed as a protrusion of the wall of the aerosol chamber 12.

FIGS. 9a and 10a again show cross-sectional embodiments in which the impingement element 10 is designed separately from the receptacle 5 and sleeve 7. In these embodiments, the impingement element is integrated into the insert 21, which—in FIGS. 9a and 10a respectively from the right—is inserted into the mouthpiece 18. The direction of insertion according to the above definitions is therefore from right to left in FIGS. 9a and 10a.

The insert 21 from FIG. 9a is shown in FIG. 9b without the remaining inhalation actuator in the top view in the direction of insertion (i.e., from the right in FIG. 9a), and in FIG. 9c in a corresponding rear view, i.e., looking in the direction opposite to the direction of insertion (from the left in FIG. 9a). Similarly, the insert 21 from FIG. 10a is shown in FIG. 10b without the remaining inhalation actuator in the top view in the insertion direction (i.e., from the right in FIG. 10a), and in FIG. 10c in a corresponding rear view, i.e., looking in the direction opposite to the insertion direction (from the left in FIG. 10a).

FIG. 10d shows an alternative insert 21 in the rear view analogous to FIG. 10c. In FIG. 10e, this alternative insert 21 is shown in a side view, i.e., from below (or from above—which makes no difference due to symmetry) in FIGS. 10d and 10f. The cross-sectional view of the alternative insert 21 in FIG. 10f is shown analogously to the cross-sectional view in FIG. 10a.

Via the neck 23, the liquid inhalate from the inhalate nozzle 9, from the nozzle discharge opening of which it emerges, can be sprayed against the impingement element 10 through the inhalate inlet opening 24 in the wall 25 of the insert 21, which is arranged transversely to the direction of insertion. The impingement element 10 is held in place by struts 26 integral with the insert 21. The wall 25 constitutes the rear wall of the aerosol chamber 12.

Laterally, the aerosol chamber 12 is delimited by the oval tube 27a (in FIG. 9a, 9b) or 27 (in FIGS. 10a, 10b), which also serves as a centering means for centering the insert 21 in the mouthpiece 18. In FIG. 9a, the oval tube 27a is extended against the insertion direction over the oval tube 27b of the further insert 31, which is also inserted into the mouthpiece 18. The tube 27b in turn serves as a centering means for centering the further insert 31 in the mouthpiece 18.

The further insert 31 is shown in FIG. 9d looking in the opposite direction to the direction of insertion (from the left in FIG. 9a). Together with the insert 21, it forms the aerosol chamber 12.

The asymmetrical rim 28 locally constricts the aerosol outlet 11 from the aerosol chamber 12, i.e., it limits its outlet area 14. When the inhalation actuator is held upside down as intended, as shown in FIGS. 9a and 10a, an inhalate collection chamber 17 is also formed by means of the rim 28 and the wall 25, which prevents excess inhalate dripping from the impingement element 10 from running out of the opening of the mouthpiece 18.

Air can flow into the aerosol chamber 12 through the air inlet 13. In the insert 21 of FIGS. 9b, 9c, 10b and 10c, the air inlet 13 is formed in the form of openings in the wall 25, that is, the rear wall of the aerosol chamber 12. In the alternative insert 21 of FIGS. 10d, 10e, the air inlet 13 consists of wall interruptions in a section of the tube 27 that is not completely covered by the wall of the mouthpiece 18.

The inhalation devices in FIGS. 9a and 10a also comprise an inhalation device 1 having a pressure container 2, a metering valve unit 3 and a discharge nipple 4. The metering valve unit 3 is again operable by either gripping the sleeve 7 and pressing the pressure container 2 downward with the thumb, or preferably by holding the entire assembly between the thumb and index finger and pressing it together, the thumb being placed at the bottom of the actuator and the index finger at the top of the base of the pressure container 2. For support, a thumb recess (not shown) can again advantageously be provided herein in the head portion of the actuator 6.

The propellant pressure in the pressure container 2, the diameter of the nozzle discharge opening and the distance between the nozzle discharge opening and the impingement element 10 are in turn matched to each other and to the physical properties of the inhalate in such a way that, after emerging from the nozzle discharge opening, the inhalate liquid breaks up into droplets by free jet disintegration, which then impinge on the impingement element 10. The impact on the impingement element 10 further atomizes and decelerates the inhalate droplets.

1. An inhalation actuator, comprising
a receptacle for receiving a discharge nipple of an inhalate delivery device;
an inhalate nozzle for spraying inhalate from at least one nozzle discharge opening;
an inhalate feed for supplying inhalate from the discharge nipple to the inhalate nozzle;
an impingement element onto which inhalate can be sprayed from the nozzle discharge opening in a straight line; and
an aerosol chamber having an internal volume to which inhalate sprayed from the inhalate nozzle is deliverable,
wherein the aerosol chamber comprises an aerosol outlet having a local constriction for outflow of aerosol from the aerosol chamber and an air supply separate from the inhalate nozzle and the aerosol outlet.

2. The inhalation actuator according to claim 1, further comprising:
an insert forming at least one wall of the aerosol chamber and supporting the impingement element.

3. The inhalation actuator according to claim 2, wherein the insert forms the aerosol chamber.

4. The inhalation actuator according to claim 1, wherein the receptacle, the inhalate feed and the aerosol chamber are made integral with each other.

5. The inhalation actuator according to claim 1, wherein the at least one nozzle discharge opening and the aerosol outlet are arranged relative to each other such that inhalate cannot flow in a straight line from the nozzle discharge opening to the aerosol outlet.

6. The inhalation actuator according to claim 1, wherein the main discharge direction of the at least one nozzle discharge opening and the main discharge direction of the aerosol outlet are arranged at least one of offset relative to one another or are at an angle greater than zero and less than 180° relative to one another,
the main discharge direction being defined as a perpendicular to the center of area of the smallest through-flowable area of the nozzle discharge opening or of the aerosol outlet, respectively.

7. The inhalation actuator according to claim 6, wherein the main discharge direction of the at least one nozzle discharge opening and the main discharge direction of the aerosol outlet are at an angle greater than 29° and less than 151° to each other.

8. The inhalation actuator according to claim 1, wherein the air supply is provided with an air supply valve.

9. The inhalation actuator according to claim 1, wherein the aerosol outlet is provided with an aerosol outlet valve.

10. The inhalation actuator according to claim 1, further comprising a mouthpiece.

11. The inhalation actuator according to claim 10, wherein the aerosol outlet is arranged between the aerosol chamber and the mouthpiece.

12. The inhalation actuator according to claim 1, further comprising a collecting device for collecting inhalate that it at least one of dripping or flowing off from the impingement element or precipitating in the aerosol chamber.

13. The inhalation actuator according to claim 12, wherein the collecting device comprises an absorbent piece of material.

14. The inhalation actuator according to claim 1, a maximum extension of which extends parallel to the discharge nipple inserted into the receptacle as intended.

15. The inhalation actuator according to claim 1, wherein at least 10 percent of the internal volume of the aerosol chamber is, with respect to a direction defined by a straight connecting line from the nozzle discharge opening to the center of area of a discharge area of the aerosol outlet, located behind the nozzle discharge opening.

16. An insert for insertion into a mouthpiece of an inhalation actuator, said insert comprising:
centering means for centering the insert in the mouthpiece;
a wall arranged transversely to the insertion direction, said wall being arranged in front of the centering means in the insertion direction;
an inhalant inlet opening provided in the wall;
an impingement element which is arranged in front of the inhalate inlet opening in [[the]] a direction opposite to the insertion direction; a free-flow area arranged transversely to the insertion direction next to the impingement element that can be flowed through freely in the insertion direction;
an air inlet arranged in front of the free-flow area in the insertion direction; and
an aerosol outlet arranged opposite to a direction of insertion in front of the free-flow area.

17. The insert according to claim 16, wherein the centering means are integrally formed with a continuous or interrupted tube, a longitudinal direction of the tube corresponding to the insertion direction.

18. The insert according to claim 17, wherein the tube comprises on its side opposite, in longitudinal direction, of said wall an edge constricting the aerosol outlet.

19. The insert according to claim 17, wherein the air inlet is at least partially formed as an interruption in the tube.

20. The insert according to claim 16, wherein the air inlet is at least partially implemented in the wall.

21. The insert according to claim 16, wherein a cross-sectional area of the aerosol outlet through which air can flow in the direction of insertion is smaller than the free-flow area next to the impingement element.

22. The insert according to claim 16, further comprising an inhalation inlet neck in front of the inhalation inlet opening in the insertion direction.

23. An inhalation actuator comprising;
a receptacle for receiving a discharge nipple of an inhalant delivery device;
an inhalate nozzle for spraying inhalate from at least one nozzle discharge opening;
an inhalate feed for supplying inhalate from the discharge nipple to the inhalate nozzle;
a mouthpiece disposed in front of the at least one nozzle discharge opening, opening; and
an insert inserted into the mouthpiece according to claim 16 such that there is a straight flow path from the at least one nozzle discharge opening through the inhalate inlet opening to the impingement element.

24. An inhalation device comprising an inhalation actuator according to claim 1 and an inhalate reservoir.

25. The inhalation device according to claim 24, wherein the inhalate reservoir comprises a pressurized container.

26. The inhalation device according to claim 25, wherein the pressurized container is provided with a metering valve.

27. The inhalation device according to claim 25, wherein the discharge nipple is oriented in a direction of a longitudinal axis of the pressure container, and
at least a predominant part of the internal volume of the aerosol chamber is located within a projection of the pressure container in the direction of its longitudinal axis.

28. An inhalation device, comprising an inhalation reservoir comprising a pressure vessel having a longitudinal axis and a discharge nipple oriented in a direction of the longitudinal axis, axis; and
an inhalation actuator, comprising:
a receptacle for receiving the discharge nipple,
an inhalant nozzle for spraying inhalant from at least one nozzle discharge opening,
an inhalate feed for supplying inhalate from the discharge nipple to the inhalate nozzle,
an impingement element onto which inhalate can be sprayed from the nozzle discharge opening in a straight line, and
an aerosol chamber having an internal volume to which inhalate sprayed from the inhalate nozzle is deliverable,
wherein the aerosol chamber comprises an aerosol outlet for discharging aerosol from the aerosol chamber and an air supply separate from the inhalate nozzle and the aerosol outlet, and
wherein at least a majority of an interior volume of the aerosol chamber is disposed within a projection of the pressure vessel in the direction of its longitudinal axis.

29. The inhalation device according to claim 28, wherein the at least one nozzle discharge opening and the aerosol outlet are arranged relative to each other such that inhalate cannot flow in a straight line from the nozzle discharge opening to the aerosol outlet.

30. The inhalation device according to claim 28, wherein the main discharge direction of the at least one nozzle discharge opening and the main discharge direction of the aerosol outlet are arranged at least one of offset from each other or are at an angle greater than zero and less than 180° to each other,
the main discharge direction being defined as a perpendicular to the center of area of the smallest through-flowable area of the nozzle discharge opening or of the aerosol outlet, respectively.

31. The inhalation device according to claim 30, wherein the main discharge direction of the at least one nozzle discharge opening and the main discharge direction of the aerosol outlet are at an angle greater than 29° and less than 151° to each other.

32. The inhalation device according to claim 28, wherein the receptacle, the inhalate feed and the aerosol chamber are formed integral with each other.

33. The inhalation device according to claim 28, wherein the air supply is provided with an air supply valve.

34. The inhalation device according to claim 28, wherein the aerosol outlet is provided with an aerosol outlet valve.

35. The inhalation device according to claim 28, which comprises a mouthpiece, wherein the aerosol outlet is arranged between the aerosol chamber and the mouthpiece.

36. The inhalation device according to claim 28, which comprises a collecting device for collecting inhalate that is at least one of dripping or flowing off from the impingement element or precipitating in the aerosol chamber.

37. The inhalation device according to claim 36, wherein the collecting device comprises an absorbent piece of material.

38. The inhalation device according to claim 28, wherein the maximum extension of the inhalation actuator extends parallel to the discharge nipple inserted into the receptacle as intended.

39. The inhalation device according to claim 28, wherein at least 10 percent of the internal volume of the aerosol chamber is located behind the nozzle discharge opening with respect to a direction defined by a straight connecting line from the nozzle discharge opening to the center of area of an aerosol discharge area.

40. The inhalation device according to claim 24, which is configured such that inhalate exiting the at least one nozzle discharge opening breaks up into droplets prior to impinging on the impingement element.
